(19) United States
(12) Patent Application Publication
(10) Pub. No.: US 2026/0315066 A1
(43) Pub. Date: Oct. 15, 2026

(54) BIFURCATED LOOPED SUTURE PASSER

(71) Applicants: Smith & Nephew, Inc., Memphis, TN (US); Smith & Nephew Asia Pacific Pte. Limited, Singapore (SG)

(72) Inventors: Benjamin Michael Hall, Roslindale, MA (US); Marc Joseph Balboa, Hopkinton, MA (US)

(21) Appl. No.: 19/556,840

(22) Filed: Mar. 4, 2026

Related U.S. Application Data
(60) Provisional application No. 63/769,260, filed on Mar. 10, 2025.

(51) Int. Cl. A61B 17/04

(52) U.S. Cl. CPC A61B 17/0482

(57) ABSTRACT

A braided suture for passing multiple sutures through an opening in a device or tissue. The braided suture includes a plurality of strands braided together, including a first plurality and a second plurality of strands. At a terminal end of the base length portion, the first plurality and the second plurality of strands split to define a first braided leg and a second braided leg. The first braided leg defines a first closed loop and the second braided leg defines a second closed loop, both closed loops axially offset from each other along the braided suture.

### CROSS REFERENCE TO RELATED APPLICATIONS

This application claims the benefit to U.S. Provisional App. No. 63/769,260, filed Mar. 10, 2025, titled “BIFURCATED LOOPED SUTURE PASSER”, herein incorporated by reference, in its entirety.

### FIELD

The present disclosure relates to braided suture constructs and associated methods for repairing tissue. More specifically, this disclosure relates to a braided suture construct with diverging looped ends for passing multiple sutures.

### BACKGROUND

In the field of arthroscopy, it is often necessary to shuttle or pass a plurality of sutures (or equivalent flexible members) through tissue and/or openings in surgical devices, such as anchors, implants, or instruments. When multiple sutures are passed, the combined bulk of the sutures and the suture-passing tool can become a limiting factor in the design of such devices. For example, in FIG. 1A an example anchor 100 (simplified for ease of discussion) is shown, with an opening 105. During the procedure a plurality of sutures 50a, 50b and 50c may be connected to a device, such as anchor 100, using snare 120. These sutures may include sutures, suture tape, flexible wire, or a mixture thereof. The size of opening 105, in this example case must be sized to allow the thickness of a folded over thickness of the plurality of sutures as well as the snare loop thickness. The highest volume portion is where the snare loop and sutures link, creating a volume including two diameters (2x”D”) of the snare loop and two diameters of each suture (50a, 50b, 50c), that now needs to be drawn through opening 105. If the opening 105 is only sized to fit one suture (50a for example), the increased bulk from the additional sutures (50b, 50c for example) may significantly raise the force required to pass the sutures. Alternatively, it may necessitate a larger opening 105 through which the sutures must pass, thereby increasing the overall size of the device.

When multiple sutures are passed through soft tissue, the combined bulk of the multiple sutures and the passing tool may create an opening through the soft tissue, larger than desired through the tissue. Large openings may compromise tissue integrity and frustrate effective tissue repair. As current devices and techniques increasingly seek to minimize device size and tissue disruption, there remains a need for a multi- suture-passing solution that reduces passing loads while maintaining a small passage profile.

Serial or staged passing of sutures has been disclosed in US20220233302, commonly owned and herein incorporated by reference in its entirety. The multiple sutures (50a, 50b, 50c) may then be individually threaded, through its own dedicated loop, allowing each suture 50a, 50b, 50c to be passed serially, reducing the maximum volume passed through the opening 105. For example, disclosed in this commonly owned application and shown in FIG. 1B is a suture passer 140 with two axially spaced loops 145a, 145b which may be formed by altering a weaving or braiding pattern during manufacture of the suture passer 140 to form a bifurcation 144 or divergence followed by a convergence 146. The inventors have found that braided convergence 146 is a weak connection, and that tension from the suture to be passed tends to reopen and unbraid the converging braids at convergence 146, making passing with the converging construct unreliable. Also disclosed in US20220233302 is a suture passer 160, shown in FIG. 1C, with two axially spaced loops 165a, 165b which may include a spliced length 166 separating the two loops 165a, 165b. However, the thickness or diameter of the loops of the passer 160 is doubled in this embodiment, adding bulk to the passer 160 and therefore also requiring a larger opening 105. Therefore, there remains a need for a sturdy construct, resistant to unbraiding, for passing multiple sutures, that maintains a low passing loads while maintaining a small passage profile. Such a solution may improve procedural efficiency, precision, and ergonomics for the surgeon while minimizing potential tissue damage.

### DEFINITIONS

Described herein are suture passing systems.

The term “suture” may include traditional sutures, that may be either hollow or may include braids along their core, or a combination of both, unless were specified. The term “suture” may include equivalent flexible members, such as but not limited to suture tape or flattened suture and may in some cases be cable, ribbon or wire.

“Bifurcation” is intended to mean a split or divergence while braiding strands of a suture, to result in two braided strand lengths. Bifurcation may include an uneven or even split, where the number of strands in each of the two braided strand lengths may be the same or unevenly split, such that one length may have more strands than the other.

### SUMMARY

Described herein is a passing or shuttling suture braided to form bifurcated lengths terminating with loops, the loops may be staged or axially offset from each other. This may reduce overall intraoperative passing loads and may decrease the size of an opening required to pass multiple sutures therethrough.

An example braided suture embodiment for passing multiple sutures through an opening in a device or tissue embodiment disclosed herein includes a suture having a base length portion formed of a plurality of strands braided together, including a first plurality and a second plurality of the plurality of strands. At a terminal end of the base length portion, the first plurality and the second plurality of strands split to define a first braided leg and a second braided leg, braided separately from each other. The first braided leg is formed into a first closed loop and the second braided leg includes a second closed loop, both closed loops offset from each other relative to the base length portion terminal end.

In some example embodiments, the base length portion may be preassembled to the opening of a device. The first and second closed loops may be splice loops formed by the first and second braided legs. The first leg may have a terminal end that is fixed to the second leg to form the first closed loop. The first braided leg may be braided for a first maximum length and wherein the second braided leg is braided for a second maximum length, longer than the first maximum length. The second plurality of strands may be greater than the first plurality of strands. The second plurality of strands may be more than double the first plurality of strands. The second plurality of strands may bifurcate to define a third braided leg braided separately from the first and second braided legs, the third braided leg may define a third closed loop. The third closed loop may be axially further along the braided suture from the base length portion terminal end than both the first and second closed loop. The third plurality of strands may be greater than the first plurality of strands.

Another example braided suture embodiment for passing multiple sutures serially through an opening in a device or tissue disclosed herein includes a suture construct with a base length portion formed of a plurality of strands braided together. The plurality of strands includes a first plurality, a second plurality and a third plurality of strands, wherein at a terminal end of the base length portion, the first plurality separates from the second and third plurality of strands to define a first braided leg having a free end, the second and third plurality of strands defining a second braided leg. At a location axially spaced along the braided suture from the terminal end, the second plurality of strands separates from the third plurality of strands to define a third braided leg including the second plurality of strands, and a fourth braided leg including the third plurality of strands, and wherein the first braided leg forms a first closed loop and the third braided leg forms a second closed loop.

In some example embodiments, the base length portion may be preassembled through the opening of the device. The first and second closed loops may be splice loops. The first braided leg may have a terminal end that is fixed to the second braided leg to form the first closed loop. The third braided leg may have a terminal end that is fixed to the fourth braided leg to form the second closed loop. The second plurality of strands may be greater than the first plurality of strands. The first and third plurality of strands may have the same number of strands. The fourth braided leg may form a third closed loop, axially further along the braided suture from the base length portion terminal end than both the first and second closed loop.

Another example braided suture embodiment for passing multiple sutures serially through an opening in a device or tissue disclosed herein includes a suture base length portion formed of a plurality of strands braided together, the plurality of strands including a first plurality, a second plurality and a third plurality of strands. At the terminal end of the base length portion, the first plurality of strands separates from the second and third plurality of strands to define a first braided leg, the second and third plurality of strands defining a second braided leg. At a location axially spaced along the braided suture from the terminal end, t second plurality of strands separates from the third plurality of strands to define a third braided leg including only the second plurality of strands and a fourth braided leg including the third plurality of strands. The first braided leg forms a first closed loop and the third braided leg forms a second closed loop, the first braided leg having less strands than the second braided leg.

### BRIEF DESCRIPTION OF THE DRAWINGS

The disclosure will be more fully understood by reference to the detailed description, in conjunction with the following figures, wherein:

FIG. 1A schematically shows an example prior art system used to pass multiple sutures;

FIGS. 1B-1C schematically show example prior art suture passers;

FIGS. 2A-2C schematically shows an example method of forming a bifurcated suture passing construct, in accordance with this disclosure;

FIG. 2D schematically shows a close up of one of the suture loops snaring a suture, in accordance with this disclosure;

FIGS. 3A schematically shows another example bifurcated suture passing construct, in accordance with this disclosure;

FIG. 3B schematically shows a close up of one of the suture loops snaring a suture, in accordance with this disclosure;

FIG. 4A schematically shows another example suture passing construct, in accordance with this disclosure;

FIG. 4B schematically shows the example suture passing construct of FIG. 4A with the sutures assembled thereto, in accordance with this disclosure; and

FIG. 5 illustrates an example suture passing construct assembled to a device, in accordance with this disclosure.

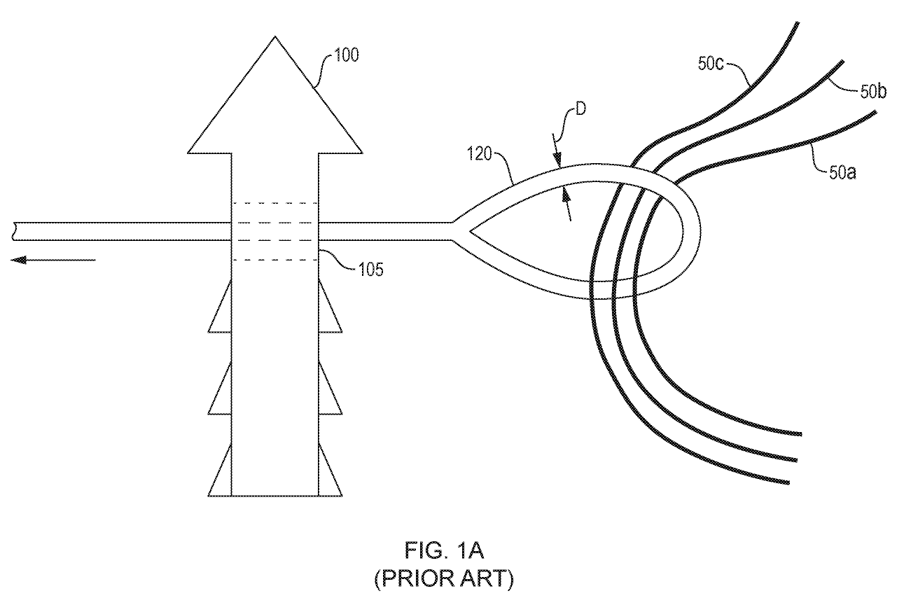

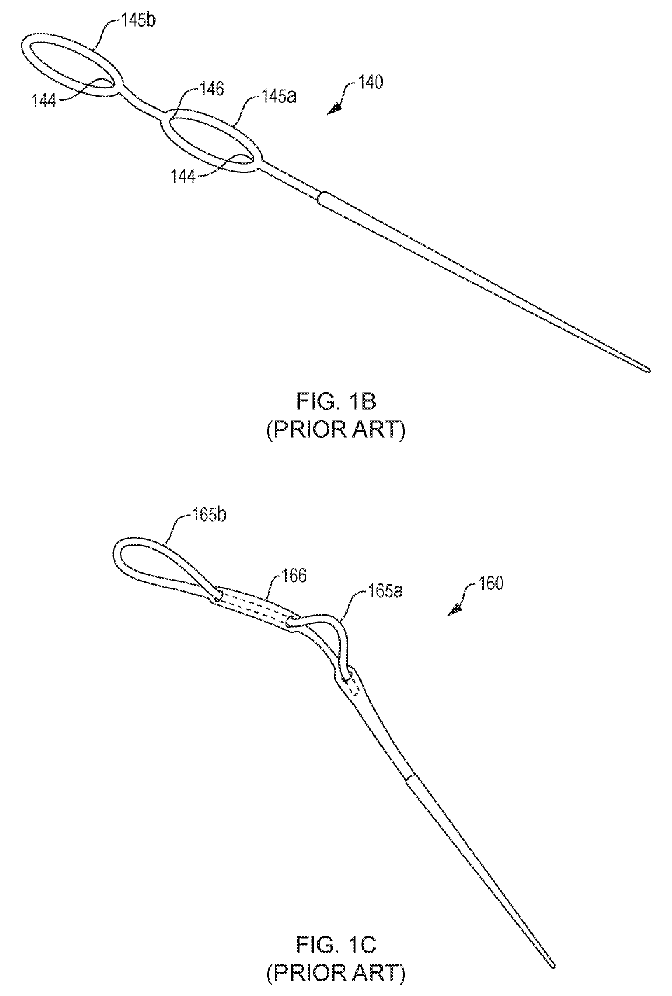

### DETAILED DESCRIPTION

In the description that follows, like components have been given the same reference numerals, regardless of whether they are shown in different examples. To illustrate example(s) in a clear and concise manner, the drawings may not necessarily be to scale, and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

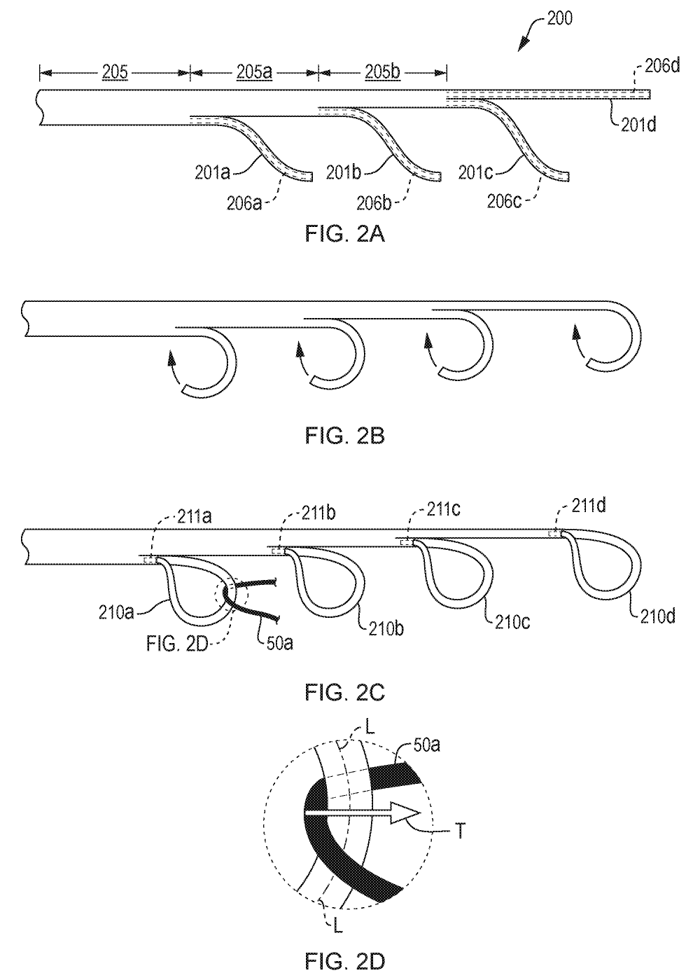

FIGS. 2A–2C illustrate an example method for forming a bifurcated looped suture passer 200 that enables passage of multiple sutures with reduced passing load and improved loop reliability. Staging the passing of the sutures reduces the load to pass them through a device opening 105 or tissue. Maintaining a smaller passing loop bulk also may reduce the passing load. Forming loops such that the tension from the passing suture is perpendicular to the major braiding orientation may also improve loop reliability.

FIG. 2A illustrates a suture 200 with three bifurcations axially separated along the length of the suture 200, forming, by example, 4 diverging legs 201a, 201b, 201c and 201d. This is an example number of bifurcations, and the disclosure is not limited to three. At each bifurcation, during the fabrication by braiding, the carrier count may be rearranged by a percentage of the total. Each leg 201a, 201b, 201c and 201d is a braided leg, and may be arranged to create a passing loop. The staged (axially spaced) arrangement of this construct, along with the reduced passing suture size (as a result of the bifurcation leading to reduce number of braids) reduces passing loads due to reduced bulk when passing. Another benefit of this passing suture is that it does not require multiple passing steps for the surgeon.

Once braided as shown in FIG. 2A, passing loops 210a, 210b, 210c, and 210d may then be formed, as shown in FIGS. 2B and 2C. Each leg 201a, 201b, 201c and 201d may be braided to form a hollow core 206a, 206b, 206c, 206d in order to form a spliced loop 210a, 210b, 210c, and 210d, formed by inserting the corresponding end 211a, 211b, 211c and 211d of each leg 201a, 201b, 201c, and 201d into a corresponding hollow core 206a, 206b, 206c, 206d for a short distance. This short distance may be sufficient to retain the end 211a, 211b, 211c and 211d in place with tension while passing. It is preferable that each leg 201a, 201b, 201c, and 201d extend into a hollow core 206a, 206b, 206c, 206d of the same leg, given the reduced number of strands. This reduced number of strands forms a smaller hollow core, more likely to cinch around the corresponding ends 211a, 211b, 211c and 211d. Stated another day, should end 211a be spaced into the base portion 205, it is unlikely a splice into a larger size suture will provide sufficient hold of the end 211a. Splice ends 211a, 211b, 211c and 211d may be fixed in place via friction along the splice, and may be further bolstered via adhesives, heat sealing or an added stitch.

Consider now, that each leg 201a, 201b, 201c, and 201d has a longitudinal central axis L-L, shown in FIG. 2D. Braiding generally extends along the longitudinal axis L-L. Using a splice positions the applied tension T from the suture (50a shown for example) approximately perpendicularly to the longitudinal central axis and approximately perpendicular to the braid orientation, at the location where tension T is applied by the suture 50a. Stated another way, the braiding pattern generally extends along the longitudinal axis of each leg, or at an acute angle thereto, while the majority of the tension applied by the suture 50a is oriented substantially perpendicular to the longitudinal axis of the leg. This may mitigate unbraiding or unravelling of loops 201a, 201b, 201c, and 201d.

Each leg 201a, 201b, 201c and 201d may have the same number of strands. For example, the base suture 205 may include 32 braided strands, that may or may not have a hollow core. To maintain a smaller size, it may preferably be absent a lumen. Each leg 201a, 201b, 201c and 201d may include 8 strands. Stated another way base 205a may include 24 strands, and 205b 16 strands. In other example embodiments, each leg may include different number of strands. For example, leg 201a may include only 6 strands out of a total of 32 strands, to reduce the number of strands in the first loop 210a, keeping a larger base portion (205, 205a, 205b) along a longer portion of the suture 200 and stronger suture for a longer distance. Subsequent legs 201b and 201c may include 8 strands, leaving 10 strands in the final leg 201d. While each leg may include a hollow core, each base portion 205, 205a and 205b may be braided to be absent a lumen or hollow core, allowing for a more dense and smaller OD base portion.

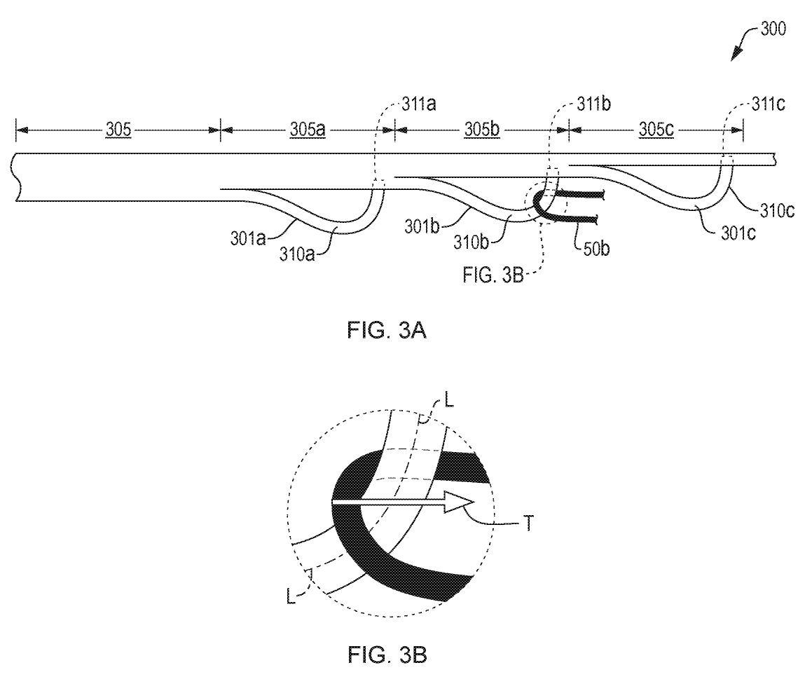

Another example embodiment 300 is represented in FIG. 3A. Embodiment 300 may be braided in a similar manner to suture 200, as shown in FIG. 2A, however the loops 301a, 301b, 301c may be formed in different orientation, coupling directly to an axially adjacent base portion 305a, 305b, 305c etc. Ends 311a, 311b and 311c may be inserted into the base portion 305a, 305b, 305c and fixed via stitching, adhesive or heat sealing for example. In this embodiment, legs 301a, 301b, 301c may be absent a lumen, with strands extending through the entire leg cross section allowing for a denser smaller diameter suture leg 301a, 301b, 301c, and reduced passing loads.

Compared to embodiments 200, embodiment 300 may have a reduces double loop thickness of each loop, further reducing the passing load. Loops 310a, 301b, 301c do not double back on themselves, compared to the loops in embodiment 200. Legs 301a, 301b, 301c may also be more densely braided, compared to legs in embodiment 200, as they do not require a hollow core. Similar to the previous embodiment, and illustrated in FIG. 3B, the tension from the passing suture is close to perpendicular to the leg longitudinal axis L-L and braiding orientation, which mitigates unbraiding or unravelling of loops 301a, 301b and 301c.

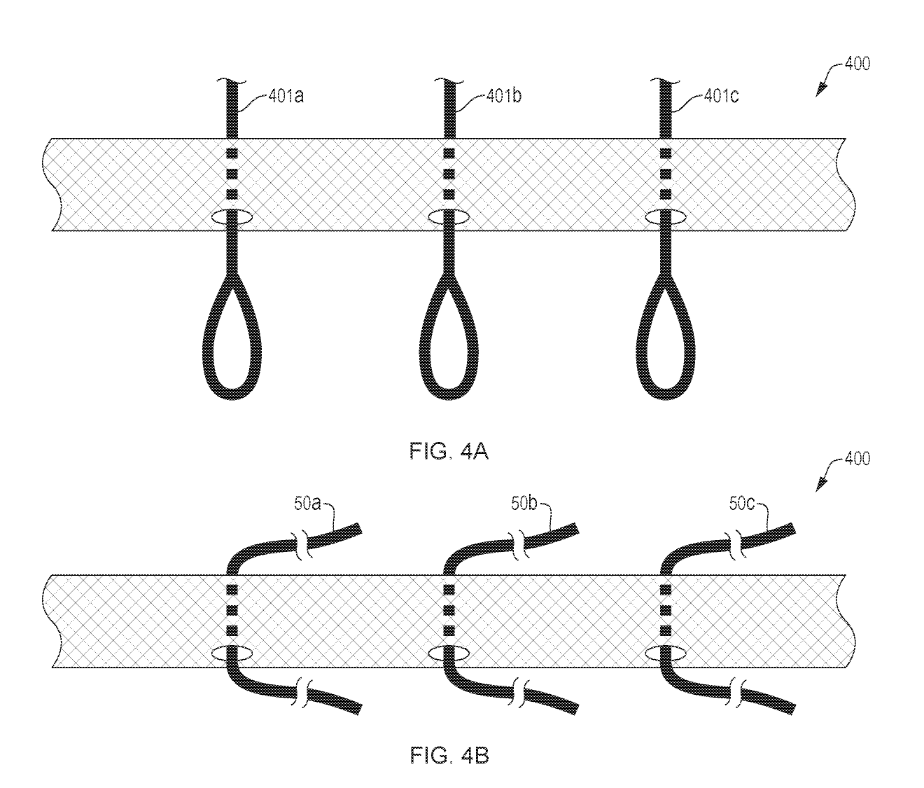

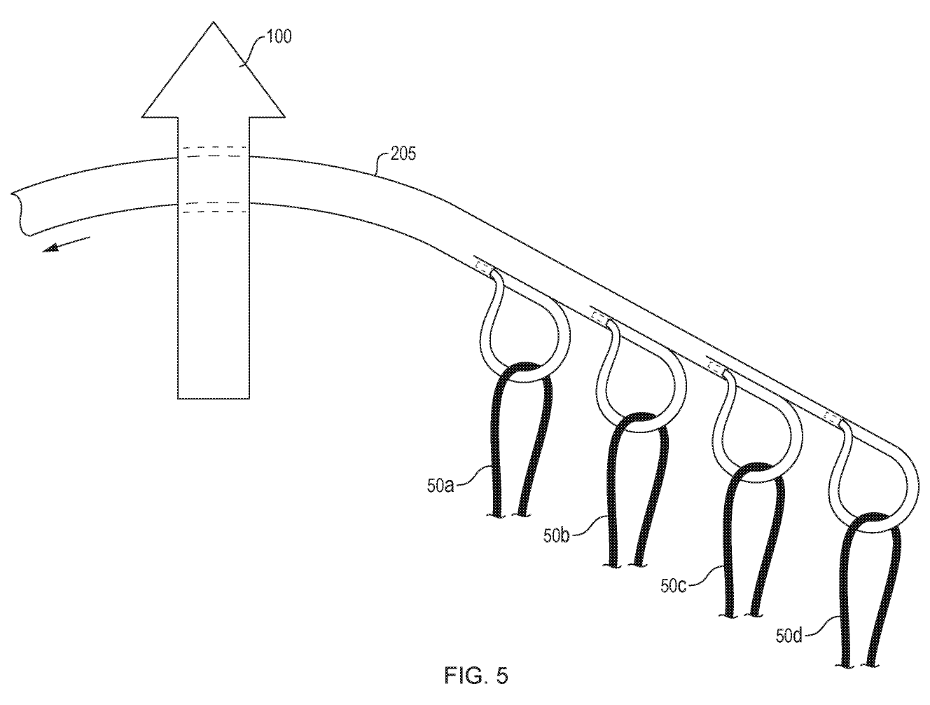

Another example embodiment 400 is represented in FIG. 4. Embodiment 400 may include a single, non-bifurcated braid with a plurality of suture snares 401, 401b, 401c axially spaced from each other. Each suture 50a, 50b, 50c may be separately drawn through the suture 400 via the suture snares, at axially spaced locations, as shown in FIG. 4B.

All embodiments have been shown with 3-4 axially spaced locations to locate a suture at an axially spaced location. Any number of loops/connections are envisioned. Each embodiment 200, 300, 400 may be provided pre-routed through a target opening, such as opening 105. In order to pass through tissue, each embodiment 200, 300, 400 may be provided coupled to a suture passer, such as a needle.

Any of the suture passers (200, 300, 400) disclosed herein may be provided preassembled with a device, such as anchor 100. Other devices (not shown), as would be understood by one of ordinary skill in the art, may include, but are not limited to, a needle, a cortical anchor, or an opening through an insertion tool, for example. A base length portion of the suture passer (200, 300, 400) may be located through opening 105 and, drawing base portion away from the opening 105 (as indicated by the arrow), may sequentially draw sutures 50a, 50b, 50c, 50d through the opening 105. The loops may be axially spaced such that each suture fold or bite is completely drawn through the opening 105 before a subsequent suture enters the opening 105, thereby reducing the likelihood of the sutures or loops within the opening 105 and allowing the opening 105 to remain relatively small.

One skilled in the art will realize the disclosure may be embodied in other specific forms without departing from the spirit or essential characteristics thereof. The foregoing examples are therefore to be considered illustrative in all respects rather than limiting of the disclosure described herein. The scope of the disclosure is thus indicated by the appended claims, rather than by the foregoing description, and all changes that come within the meaning and range of equivalency of the claims are therefore intended to be embraced therein.

What is claimed is:

1. A braided suture for passing multiple sutures through an opening in a device or tissue, comprising:
a base length portion formed of a plurality of strands braided together, including a first plurality and a second plurality of the plurality of strands, wherein at a terminal end of the base length portion, the first plurality and the second plurality of strands split to define a first braided leg and a second braided leg, braided separately from each other;
wherein the first braided leg defines a first closed loop and the second braided leg includes a second closed loop, both closed loops axially offset from each other relative to the base length portion terminal end.

2. The braided suture of claim 1, wherein the base length portion is preassembled to the opening of the device.

3. The braided suture of claim 1 wherein the first and second closed loops are splice loops.

4. The braided suture of claim 1, wherein the first leg terminal end is fixed the second leg to form the first closed loop.

5. The braided suture of claim 1 wherein the first braided leg is braided for a first maximum length and wherein the second braided leg is braided for a second maximum length, longer than the first maximum length.

6. The braided suture of claim 1, wherein the second plurality of strands is greater than the first plurality of strands.

7. The braided suture of claim 6, wherein the second plurality of strands is more than double the first plurality of strands.

8. The braided suture of claim 1 wherein the second plurality of strands bifurcates to define a third braided leg braided separately from the first and second braided legs, the third braided leg including a third closed loop.

9. The braided suture of claim 8 wherein the third closed loop is axially further along the braided suture from the base length portion terminal end than both the first and second closed loop.

10. The braided suture of claim 8, wherein the third plurality of strands is greater than the first plurality of strands.

11. A braided suture for passing multiple sutures serially through an opening in a device or tissue, comprising:
a base length portion formed of a plurality of strands braided together, the plurality of strands including a first plurality, a second plurality and a third plurality of strands, wherein at a terminal end of the base length portion, the first plurality separates from the second and third plurality of strands to define a first braided leg, the second and third plurality of strands braided together to define a second braided leg;
wherein the second plurality of strands separates from the third plurality of strands to define a third braided leg including the second plurality of strands, and a fourth braided leg including the third plurality of strands, the second plurality of strands separating at a location axially spaced along the braided suture from the terminal end; and wherein the first braided leg forms a first closed loop and the third braided leg forms a second closed loop.

12. The braided suture of claim 11, wherein the base length portion is preassembled through the opening of the device.

13. The braided suture of claim 11 wherein the first and second closed loops are splice loops.

14. The braided suture of claim 11, wherein the first braided leg has a terminal end that is fixed to the second braided leg to form the first closed loop.

15. The braided suture of claim 14, wherein the third braided leg has a terminal end that is fixed to the fourth braided leg to form the second closed loop.

16. The braided suture of claim 11, wherein the second plurality of strands, is greater than the first plurality of strands.

17. The braided suture of claim 11, wherein the first and third plurality of strands have the same number of strands.

18. The braided suture of claim 11 wherein the fourth braided leg forms a third closed loop, axially further along the braided suture from the base length portion terminal end than both the first and second closed loop.

19. A braided suture for passing multiple sutures serially through an opening in a device or tissue, comprising:
a base length portion formed of a plurality of strands braided together, the plurality of strands including a first plurality, a second plurality and a third plurality of strands, wherein at a terminal end of the base length portion, the first plurality separates from the second and third plurality of strands to define a first braided leg including the first plurality of strands and a second braided leg including the second and third plurality of strands;
wherein the second plurality of strands separates from the third plurality of strands to define a third braided leg including the second plurality of strands, and a fourth braided leg including the third plurality of strands, the second plurality of strands splitting at a location axially spaced along the braided suture from the terminal end; and
wherein the first braided leg forms a first closed loop, and the third braided leg forms a second closed loop, the first braided leg having less strands than the second braided leg.
